(19) United States
(12) Patent Application Publication
(10) Pub. No.: US 2008/0161892 A1
(43) Pub. Date: Jul. 3, 2008

(54) Facial Cold -Pack Holder

(76) Inventors: John Anthony Mercuro, Yorba Linda, CA (US); Scott Michael Mercuro, Aliso Viejo, CA (US)

Correspondence Address: QUICKPATENTS, INC., 32861 CALLE PERFECTO, SUITE A, SAN JUAN CAPISTRANO, CA, 92675, US

(21) Appl. No.: 11/617,442

(22) Filed: Dec. 28, 2006

(51) Int. Cl. A61F 7/10; A61F 7/08

(52) U.S. Cl. 607/109; 607/112

(57) ABSTRACT

A therapeutic device for use on a head of a patient is disclosed, and includes a chin portion adapted for receiving and substantially covering the chin of the patient. The device includes a pair of upper flexible straps, each terminating at a proximal end at the opposing sides of the chin portion, and each terminating at a distal end at an upper strap attachment means, such as a hook-and-loop type fastener. Similarly, a pair of lower flexible straps is included, each terminating at a proximal end at the opposing sides of the chin portion, and each terminating at a distal end at a lower strap attachment means. A plurality of pockets are included, each pocket comprising a front portion and a rear portion. The upper and lower straps each terminate at one of the pockets, the pocket connecting the straps to the chin portion. Each pocket includes both a closed end positioned proximate the chin portion, and an open side positioned proximate the patient's ears when the device is worn. A thermal pack is inserted into each of the pockets to deliver thermal therapy to the patient's jaws and surrounding areas.

### CROSS-REFERENCE TO RELATED APPLICATIONS

Not Applicable.

### STATEMENT REGARDING FEDERALLY SPONSORED RESEARCH AND DEVELOPMENT

Not Applicable.

### FIELD OF THE INVENTION

This invention relates to surgical devices, and more particularly to a surgical device adapted for holding an ice-pack against a patient's jaw for use after oral surgery, facial, head or neck surgery, or other trauma where such a device may be used.

### DISCUSSION OF RELATED ART

Various therapeutic bandages have been devised to maintain thermal therapy to areas of the body that have experienced trauma, such as after surgery or the like. In the area of oral surgery the prior art includes various types of bandages and wraps with the purpose of holding a cold-pack or other thermal device proximate to a person's jaws. For example, U.S. Pat. No. 3,491,761 to Baker on Jan. 27, 1970, teaches such a device. However, one drawback of such a device is that it is not specifically designed for use on the head, but rather for use in numerous areas of the body. Openings 7 in bands 4 of this type of device, therefore, are not specifically designed to line-up coincident with the ears of the user. This results in either the ears being blocked by the bands or, worse, partially blocked and uncomfortable since the user's ears may partially protrude from the openings. Further, when such a device is used as indicated the user, by opening his jaws such as to talk or take in food, will cause the length of the bands to increase, thereby causing the bands to tighten and apply excessive pressure on the jaw areas of the user. Such excessive pressure is particularly painful after surgery or other trauma, which is exactly the type of circumstance that would require the device to be used in the first place. Further, thermal packs used with such devices are not easily replaced when such devices are being worn.

U.S. Pat. No. 4,190,054 to Brennan on Feb. 26, 1980, teaches a similar device with similar drawbacks, as does U.S. Pat. No. 5,020,536 to Keen on Jun. 4, 1991. Other bandages not specifically for use with cold packs include U.S. Pat. No. 1,872,642 to Welsh on Aug. 16, 1932; U.S. Pat. No. 1,110,772 to Gunderman on Sep. 15, 1914; and U.S. Pat. No. 3,759,256 to O'Malley on Sep. 18, 1973. All such prior art devices cause undo pressure to be applied to the jaws when the user opens his mouth, since the manner in which the bands are positioned around the top of the head result in a change in length of the bands when the user opens his mouth. Further, such devices tend to be overly large and bulky, drawing significant attention to the fact that the user wearing such a device has had a serious injury. Those prior art devices that include pockets typically orient the opening of the pockets inconveniently towards the user, requiring the device to be removed completely from the head when changing thermal packs.

Therefore, there is a need for an inexpensive and relatively simple bandage for holding thermal packs against the user's jaws and surrounding areas. Such a needed device would not become tighter around the user's head when the user opens his mouth, and as such would not increase the pressure to inured areas. Further, when using such a needed device, the user's ears would be completely exposed so as to be both comfortable and not impede the user's hearing in any way. Further, such a needed device would make provision for insulating the thermal packs used therewith in all but the injured areas of the user's jaws to which the thermal therapy is to be applied. Moreover, such thermal packs would be easy to change-out without removing the device from the user's head. The present invention accomplishes these objectives.

### SUMMARY OF THE INVENTION

The present device is a therapeutic device for use on a head of a patient and includes a chin portion adapted for receiving and substantially covering the chin of the patient. In a preferred embodiment of the invention the device includes a pair of upper flexible straps, each terminating at a proximal end at the opposing sides of the chin portion, and each terminating at a distal end at an upper strap attachment means, such as a hook-and-loop type fastener. Similarly, a pair of lower flexible straps is included, each terminating at a proximal end at the opposing sides of the chin portion, and each terminating at a distal end at a lower strap attachment means.

A plurality of pockets are included, each pocket comprising a front portion and a rear portion. Preferably the front portion is a relatively think, insulating material, such as terrycloth, neoprene, or the like. The rear portion, however, is a relatively thin, non-insulating material, such as a thin layer of fabric or plastic. Each pocket may be disposed between an upper strap and a lower strap, or, in the preferred embodiment, the upper and lower straps may each terminate at one of the pockets, the pocket connecting the straps to the chin portion. Each pocket includes both a closed end positioned proximate the chin portion, and an open side positioned proximate the patient's ears when the device is worn. As such, each pocket is proximate to the patient's jaw and provides thermal therapy thereto.

In use, with the patient's chin fitted into the chin portion and the upper and lower straps each encircling the patient's head, each upper strap traversing above the person's ears and each lower strap traversing below the person's ears, a thermal pack may be inserted into each of the pockets to deliver thermal therapy to the patient's jaws and surrounding areas. Such a thermal pack may be an ice pack, a heat pack, or the like, as is known in the art, and shaped to fit inside each pocket. As such the person can open and close his mouth without stretching or otherwise putting strain on the device, the pockets maintaining position proximate the person's jaws.

The present invention is an inexpensive and relatively simple device for holding thermal packs against the user's jaws and surrounding areas. The present device does not tighten around the user's head when the user opens his mouth, and as such does not increase the pressure applied to inured areas. Further, when using such a device, the user's ears are completely exposed so that the device is both comfortable and does not impede the user's hearing. Further, the present invention provides for insulating the thermal packs used therewith in all but the areas to which the thermal therapy is to be applied. As a result, the thermal packs used with the device tend to maintain their temperature for a longer duration. Moreover, such thermal packs are easy to replace without removing the device from the user's head. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a perspective view of a preferred embodiment of the invention as fastened to a patient's head, the patient's mouth in an open position;

FIG. 2 is a perspective view of the preferred embodiment of the invention as fastened to the patient's head, the patient's mouth in a closed position;

FIG. 3 is a partial front elevational view, illustrating the preferred embodiment of the invention in a flat configuration;

FIG. 4 is a rear elevational view, illustrating the preferred embodiment of the invention in a flat configuration;

FIG. 5 is a perspective view of an alternate embodiment of the invention;

FIG. 6 is a perspective view of an alternate embodiment of the invention, illustrating a thermal face mask thereof,

FIG. 7 is a perspective view of an alternate embodiment of the invention, illustrating a pocket in a chin portion of the invention;

FIG. 8 is a perspective view of an alternate embodiment of the invention, illustrating a thermal face mask thereof;

FIG. 9A is a partially exploded perspective view of an alternate embodiment of the invention, illustrating a thermal neck mask as worn by a person and the thermal face mask of FIG. 8 as detached from the person; and

FIG. 9B is a partial perspective view of the alternate embodiment of the invention of FIG. 9A, illustrating a thermal neck mask as detached from the person.

### DETAILED DESCRIPTION OF THE PREFERRED EMBODIMENT

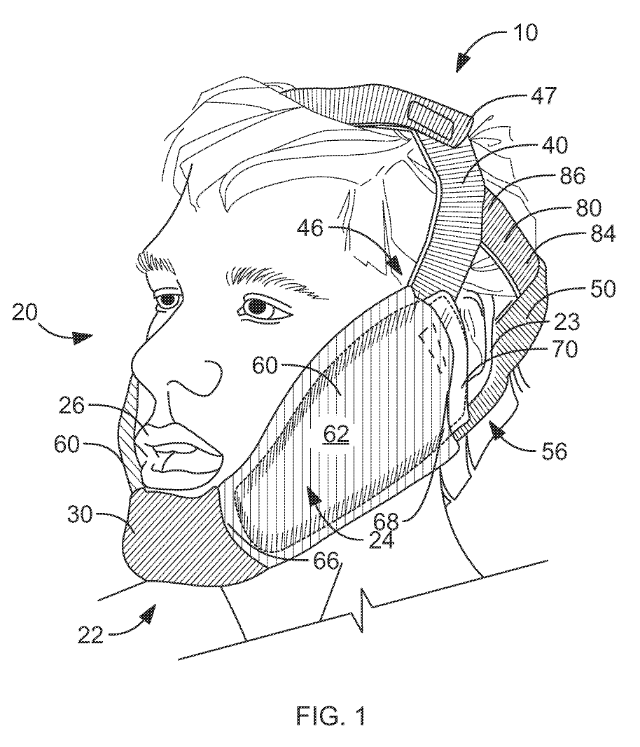

FIG. 1 illustrates a therapeutic device 10 for use on a head 21 of a patient 20. The device 10 is comprised substantially of a flexible, breathable fabric material, such as terrycloth, neoprene, or the like.

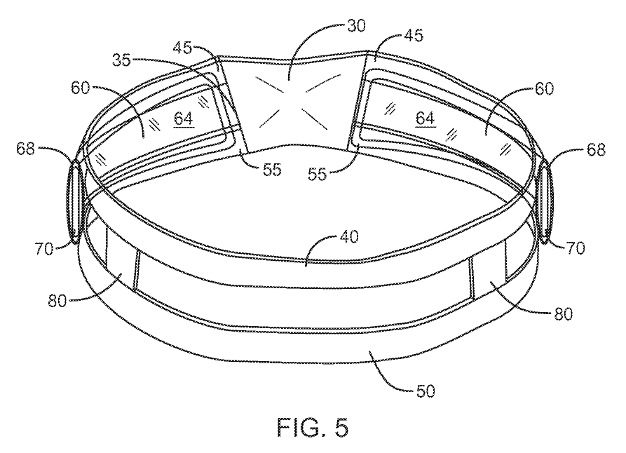

The device 10 comprises a chin portion 30 adapted for receiving and substantially covering the chin 22 of the patient. In one embodiment of the invention, illustrated in FIG. 5, an upper flexible strap 40 terminates at each end 45 at opposing sides 35 of the chin portion 30. The upper flexible strap 40 is adapted for encircling the patient's head 21 and traversing above each of the patient's ears 23.

In a preferred embodiment of the invention, illustrated in FIGS. 1-4, the device 10 includes a pair of upper flexible straps 40, each terminating at a proximal end 46 at the opposing sides 35 of the chin portion 30, and each terminating at a distal end 47 at an upper strap attachment means 48, such as a hook-and-loop type fastener 59, a mechanical snap (not shown), a mechanical buckle assembly (not shown), or any other suitable attachment means for attaching the ends of two straps 40 together, as is known in the art.

Similarly, a pair of lower flexible straps 50 is included, each terminating at a proximal end 56 at the opposing sides 35 of the chin portion 30, and each terminating at a distal end 57 at a lower strap attachment means 58.

A plurality of pockets 60 are included, each pocket 60 comprising a front portion 62 and a rear portion 64. Preferably the front portion 62 is a relatively think, insulating material, such as terrycloth, neoprene, or the like. The rear portion 64, however, is a relatively thin, non-insulating material, such as a thin layer of fabric or plastic. Each pocket 60 may be disposed between an upper strap 40 and a lower strap 50, or, in the preferred embodiment, the upper and lower straps 40,50 may each terminate at one of the pockets 60, the pocket 60 connecting the straps 40,50 to the chin portion 30. Each pocket 60 includes both a closed end 66 positioned proximate the chin portion 30, and an open side 68 positioned proximate the patient's ears 23 when the device 10 is worn. As such, each pocket 60 is proximate to the patient's jaw 24 and provides thermal therapy thereto.

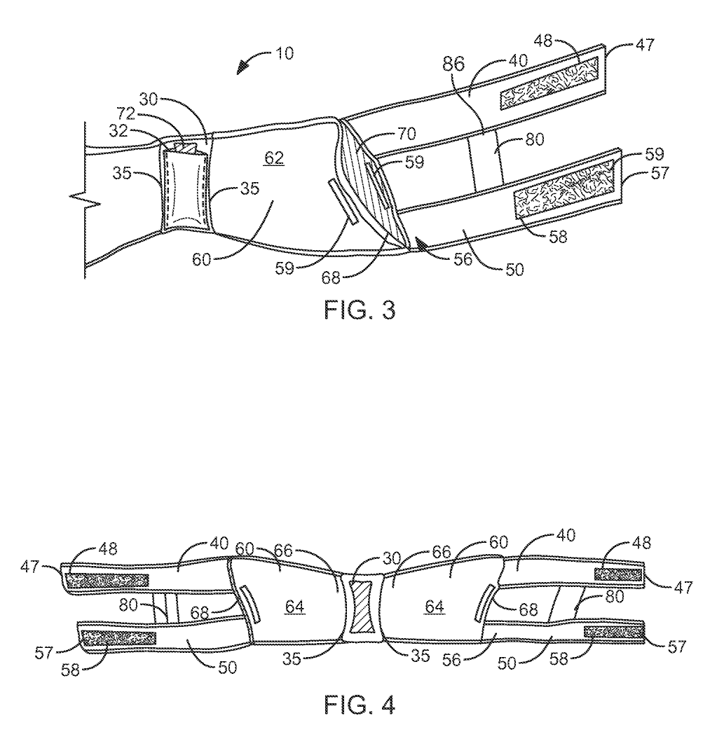

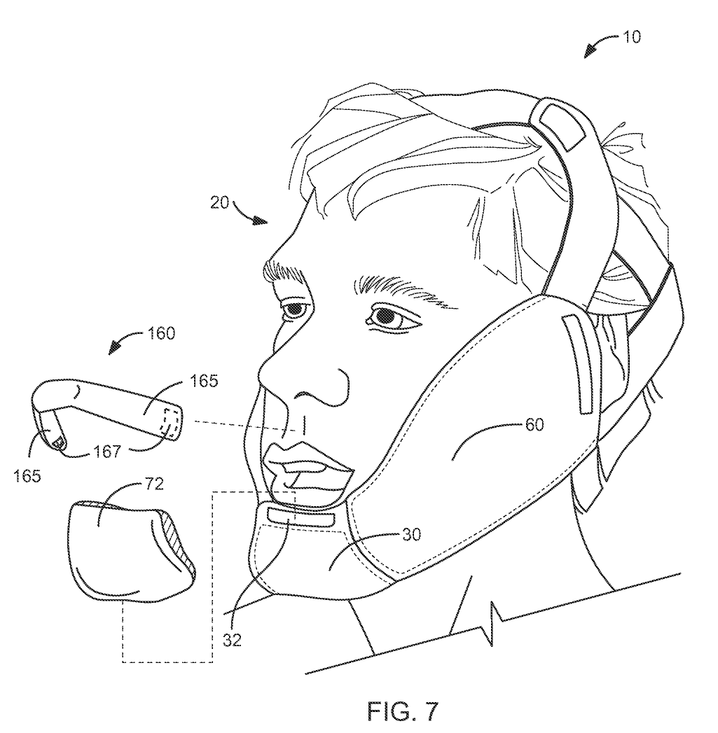

Preferably, the chin portion 30 includes a chin pocket 32 that accepts a chin thermal pack 72 for delivering thermal therapy to the patient's chin 22 (FIG. 3). Such a pocket 32 may include the advantages of features of the other pockets 60. An alternate embodiment of such a chin pocket 32 is illustrated in FIG. 7. Also illustrated in FIG. 7 is a thermally active upper lip attachment 160, which connects at either of its ends 165 with a hook-and-loop type attachment means 167 to the front portion 62 of either pocket 60.

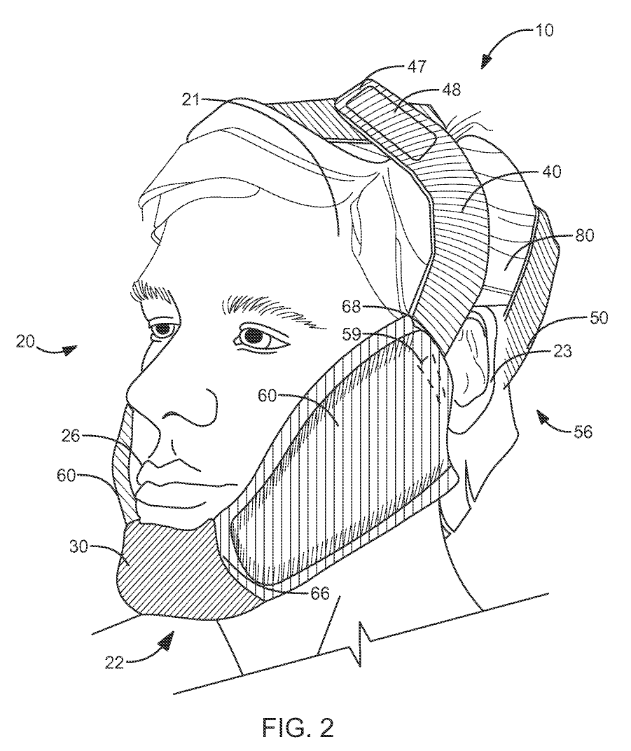

In use, with the patient's chin 22 fitted into the chin portion 30 and the upper and lower straps 40,50 each encircling the patient's head 21, each upper strap 40 traversing above the person's ears 23 and each lower strap 50 traversing below the person's ears 23, a thermal pack 70 may be inserted into each of the pockets 60 to deliver thermal therapy to the patient's jaws 24 and surrounding areas. Such a thermal pack 70 may be an ice pack, a heat pack, or the like, as is known in the art, and shaped to fit inside each pocket 60. The straps 40,50 traverse either side of the pivot point of the patient's jaws 24, and as such the person 20 can open and close his mouth 26 without stretching or otherwise putting strain on the device 10. This results in no additional pressure being applied to the person's jaws 24 during opening and closing of the mouth 26. The pockets 60 maintain their position proximate the person's jaws 24 when the person's mouth 26 is open (FIG. 1) or closed (FIG. 2).

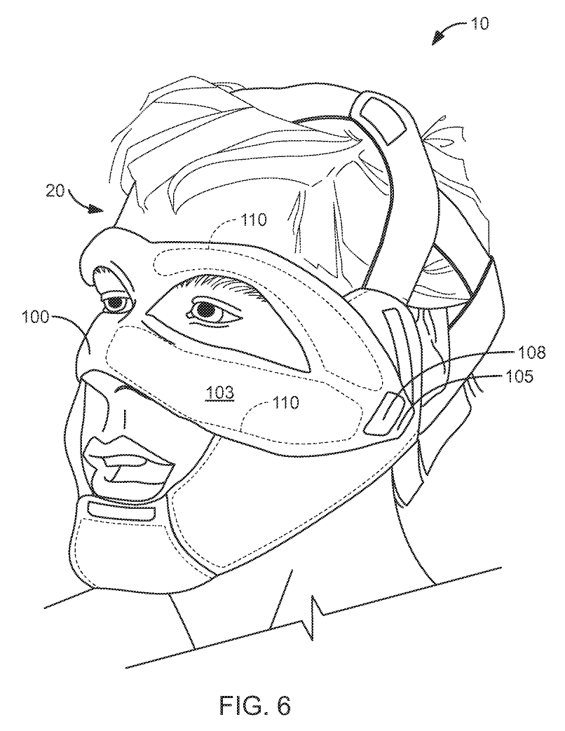

In one embodiment of the invention, illustrated in FIG. 6, a thermal eye mask 100 in further included and has two ends 105 that each include an attachment means 108, such as a hook-and-loop type fastening material. The thermal eye mask attaches across the person's eyes and nose to the front portion 62 of each pocket 60 proximate the person's temples. The thermal eye mask 100 has an elongated thermal pack 110 therein and a flexible outer covering 103, and delivers thermal therapy to the person's face around his eyes, nose, and cheek bones. In such an embodiment, the attachment means 108 is preferably a hook material and the front portion 62 of each pocket is a loop material. Alternately, the attachment means 108 may be a mechanical snap fastener (not shown).

In one embodiment of the invention, illustrated in FIG. 6, a thermal eye mask 100 is further included and has two ends 105 that each include an attachment means 108, such as a hook-and-loop type fastening material. The thermal eye mask 100 attaches across the person's eyes and nose to the front portion 62 of each pocket 60 proximate the person's temples. The thermal eye mask 100 has an elongated thermal pack 110 therein and a flexible outer covering 103, and delivers thermal therapy to the person's face around his eyes, nose, and cheek bones. In such an embodiment, the attachment means 108 is preferably a hook material and the front portion 62 of each pocket is a loop material. Alternately, the attachment means 108 may be a mechanical snap fastener (not shown).

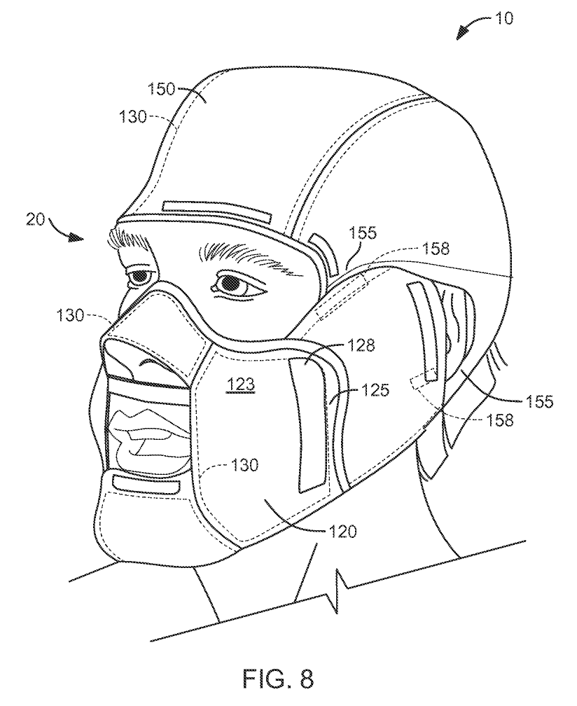

In an alternate embodiment of the invention, illustrated in FIG. 8, a thermal face mask 120 is further included and has two ends 125 that each include an attachment means 128, such as a hook-and-loop type fastening material. The face mask 120 attaches across the person's mouth and nose to the front portion 62 of each pocket 60 proximate the person's chin 22. The face mask 120 preferably includes at least one thermal pack 130 therein and a flexible outer covering 123, and delivers thermal therapy to the person's face around his mouth and nose. In such an embodiment, the attachment means 128 is preferably a hook material and the front portion 62 of each pocket is a loop material. Alternately, the attachment means 128 may be a mechanical snap fastener (not shown).

In an alternate embodiment of the invention, also illustrated in FIG. 8, a thermal head mask 150 is further included and has two ends 155 that each include an attachment means 158, such as a hook-and-loop type fastening material. The thermal head mask 150 attaches across the back of the person's head, the ends 155 each attaching to one of the pockets 60 as illustrated. The thermal head mask 150 preferably includes at least one thermal pack 130 therein and a flexible outer covering 153, and delivers thermal therapy to the top and back of the person's head. In such an embodiment, the attachment means 158 is preferably a hook material and each pocket 60 is a loop material. Alternately, the attachment means 158 may be a mechanical snap fastener (not shown).

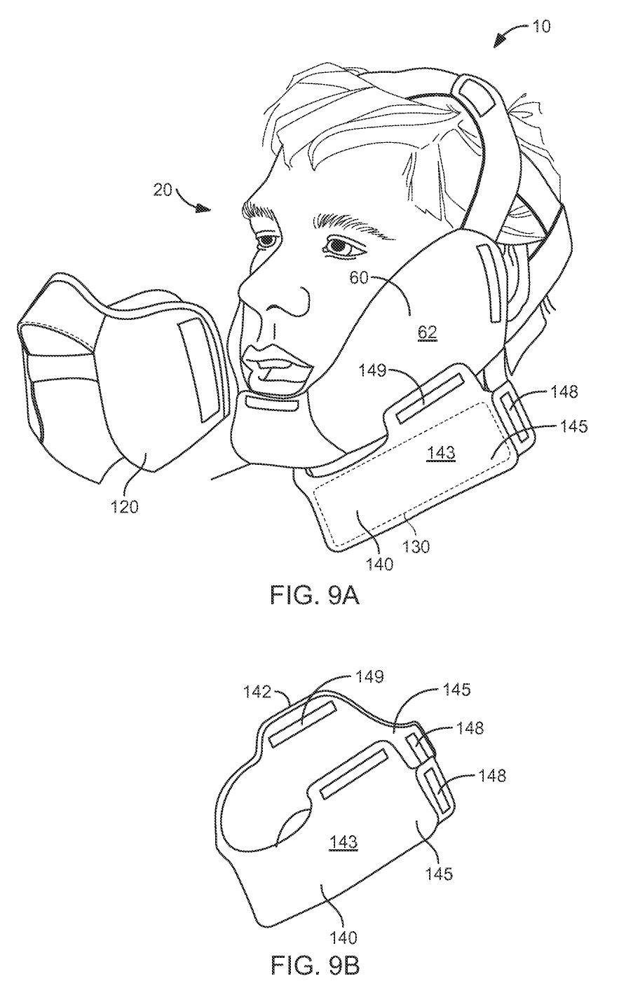

In yet another embodiment of the invention, illustrated in FIGS. 9A and 9B, a thermal neck mask 140 is further included and has two ends 145 that each include an attachment means 148, such as a hook-and-loop type fastening material. The thermal neck mask 140 attaches around the person's neck, the attachment means 148 of each side 145 mutually attaching to hold the thermal neck mask 140 in place around the person's neck. A further attachment means 149 is included on an upper edge 142 of the thermal neck mask 140 for attaching the thermal neck mask 140 to the front portion 62 of each pocket 60 proximate the person's jaw 24. As such, the thermal neck mask 140 includes at least one pocket (not shown) for holding one of the thermal packs 130 therein. The thermal neck mask 140 further includes a flexible outer covering 143, and delivers thermal therapy to the person's neck. In such an embodiment, the attachment means 149 is preferably a hook material and the front portion 62 of each pocket is a loop material. Alternately, the attachment means 149 may be a mechanical snap fastener (not shown).

While a particular form of the invention has been illustrated and described, it will be apparent that various modifications can be made without departing from the spirit and scope of the invention. For example, the type of material used with the device 10 can vary widely as needed, as can the exact number of pockets 60, and the fastening means 48,58. Accordingly, it is not intended that the invention be limited, except as by the appended claims.

What is claimed is:

1. A device for use on a patient's head, the device comprising:
a chin portion adapted for receiving the chin of the patient;
an upper flexible strap terminating at each end at opposing sides of the chin portion, the upper flexible strap adapted for encircling the patient's head and traversing above each of the patient's ears;
a lower flexible strap terminating at each end at opposing sides of the chin portion, the lower flexible strap adapted for encircling the patient's head and traversing below each of the patient's ears;
a plurality of pockets, each pocket comprising a front portion and a rear portion and being disposed between the upper and the lower straps, a closed end of each pocket being positioned proximate the chin portion, each pocket being open at a side between the upper and lower straps proximate one of the patient's ears;
whereby with the device worn by the patient, a thermal pack may be inserted into each of the pockets to deliver thermal therapy to the patient's jaws and surrounding areas.

2. The device of claim 1 further including a pair of spacer straps each fixed at one end to the lower strap and each fixed at another end to the upper strap, each spacer strap adapted for traversing between the upper strap and lower strap at a position behind one of the patient's ears, each spacer strap for maintaining a predetermined distance between the upper and lower straps around the back of the patient's head.

3. The device of claim 1 wherein the rear portion of each pocket is a relatively thin, non-insulating material, and wherein the front portion of each pocket is a relatively thick, insulating material.

4. The device of claim 1 wherein the chin portion and each strap is made from a relatively elastic material, such that the device may be stretched over the patient's head to apply the device thereto.

5. The device of claim 1 wherein each strap terminates proximate to the open end of one of the pockets, the pocket connecting the straps to the chin portion.

6. The device of claim 1 wherein each thermal pack is an ice pack.

7. The device of claim 1 wherein each thermal pack is a heat pack.

8. The device of claim 1 wherein the chin portion further includes a pocket.

9. A device for use on a patient's head, the device comprising:
a chin portion adapted for receiving the chin of the patient;
a pair of upper flexible straps, each terminating at a proximal end at opposing sides of the chin portion, and each terminating at a distal end at an upper strap attachment means, the upper flexible straps adapted for encircling the patient's head and traversing above each of the patient's ears;
a pair of lower flexible straps, each terminating at a proximal end at opposing sides of the chin portion, and each terminating at a distal end at a lower strap attachment means, the lower flexible straps adapted for encircling the patient's head and traversing below each of the patient's ears;
a plurality of pockets, each pocket comprising a front portion and a rear portion and being disposed between one of the upper and one of the lower straps, a closed end of each pocket being positioned proximate the chin portion, each pocket being open at a side between the upper and lower straps proximate one of the patient's ears;
whereby with the device worn by the patient, a thermal pack may be inserted into each of the pockets to deliver thermal therapy to the patient's jaws and surrounding areas.

10. The device of claim 9 further including a pair of spacer straps each fixed at one end to one of the lower straps and each fixed at another end to one of the upper straps, each spacer strap adapted for traversing between the upper strap and lower strap at a position behind one of the patient's ears, each spacer strap for maintaining a predetermined distance between the upper and lower straps around the back of the patient's head.

11. The device of claim 9 wherein the rear portion of each pocket is a relatively thin, non-insulating material, and wherein the front portion of each pocket is a relatively thick, insulating material.

12. The device of claim 9 wherein each strap terminates proximate to the open end of one of the pockets, the pocket connecting the straps to the chin portion.

13. The device of claim 9 wherein each thermal pack is an ice pack.

14. The device of claim 9 wherein each thermal pack is a heat pack.

15. The device of claim 9 wherein each attachment means is a mechanical snap.

16. The device of claim 9 wherein each attachment means is a hook-and-loop type fastening material.

17. The device of claim 9 wherein each attachment means is a mechanical buckle assembly.

18. The device of claim 1 further including a thermal eye mask having two ends that each include an attachment means for attaching the thermal eye mask across the person's eyes and nose to the front portion of each pocket proximate the person's temples, the thermal eye mask having an elongated thermal pack therein and a flexible outer covering.

19. The device of claim 1 further including a thermal face mask having two ends that each include an attachment means for attaching the thermal eye mask across the person's mouth and nose to the front portion of each pocket proximate the person's chin, the thermal face mask having a plurality of pockets for holding one of the thermal packs.

20. The device of claim 1 further including a thermal neck mask having two ends that each include a mutual attachment means for attaching the thermal neck mask around the person's neck, and further including attachment means on an upper edge thereof for attaching the thermal neck mask to the front portion of each pocket proximate the person's jaw, the thermal face mask having at least one pocket for holding one of the thermal packs therein.
